(19) United States
(12) Patent Application Publication
(10) Pub. No.: US 2003/0102217 A1
(43) Pub. Date: Jun. 5, 2003

(54) Method for forming multilayer coating film

(75) Inventors: Naoko Kasahara, Hiratsuka-shi (JP); Takeo Ohtani, Aichi-ken (JP); Tadayoshi Hiraki, Odawara-shi (JP)

Correspondence Address: ARMSTRONG,WESTERMAN & HATTORI, LLP, 1725 K STREET, NW., SUITE 1000, WASHINGTON, DC, 20006, US

(73) Assignee: KANSAI PAINT CO., LTD

(21) Appl. No.: 10/227,292

(22) Filed: Aug. 26, 2002

(30) Foreign Application Priority Data
Aug. 31, 2001 (JP) 2001-263647

(51) Int. Cl. C25D 1/12; G01L 1/20; C07K 1/26; G01F 1/64; C02F 1/469; C08F 2/58; C25B 7/00; G01L 9/18

(52) U.S. Cl. 204/507; 204/501

(57) ABSTRACT

The present invention provides a method for forming a multilayer coating film, comprising: applying a colored cationic electrodeposition coating composition (A) to a metal substrate by electrodeposition; applying a bright pigment-containing clear coating composition (B) to the electrodeposition coating, which is either uncured or thermally cured; and then curing the uncured electrodeposition coating and the clear coating, or the clear coating.

### BACKGROUND OF THE INVENTION

(1) Field of the Invention

The present invention relates to a method for forming a multilayer coating film.

(2) Description of Related Art

Cationic electrodeposition coating compositions have the features such as their ability to form coating films excellent in weather resistance, corrosion resistance and finish properties, and are widely used to coat metal substrates such as automobile bodies, metal parts for two-wheeled vehicles, household electrical appliances, furniture and the like.

In recent years, there have been put on the market an increasing number of products finished with a single electrodeposition coating, because of the above features of cationic electrodeposition coating compositions. Also, there has been a demand for new design with attractive visual effects for such products.

To impart new design to various products by forming cationic electrodeposition coating films thereon, techniques have been proposed for coloring an electrodeposition coating film by adding a coloring pigment to a cationic electrodeposition coating composition (Japanese Unexamined Patent Publications Nos. 1985-24400, 1985-70200, 1988-157899, etc.).

The proposed techniques, although capable of coloring the coating films, cannot give brightness to the coating films. Even if a bright pigment, such as a metallic pigment or a pearl pigment, together with a coloring pigment, is added to a cationic electrodeposition coating composition in order to give it brightness, it is difficult to impart sufficient brightness to the coating film since there is a limit to the total pigment concentration in an electrodeposition coating composition. Moreover, the techniques cannot impart three-dimensional brightness.

Accordingly, there is a strong demand for an electrodeposition coating film which has brightness, especially three-dimensional brightness, while maintaining the attributes of electrodeposition coating films, such as excellent weather resistance, corrosion resistance and finish properties.

### BRIEF SUMMARY OF THE INVENTION

An object of the present invention is to provide a method for forming, on a metal substrate, a coating film which offers new design with attractive visual effects, such as three-dimensional brightness, glitter and/or pearly luster, while maintaining the features of electrodeposition coating films, such as excellent weather resistance, corrosion resistance and finish properties.

The present inventors conducted extensive research to achieve the above object. As a result, the inventors found that the object can be achieved by applying a colored cationic electrodeposition coating composition to a metal substrate, followed by the application of a clear coating composition containing a bright pigment to the coated substrate. The invention has been accomplished based on the above novel finding.

The invention provides the following methods for forming multilayer coating films.

1. A method for forming a multilayer coating film, comprising the steps of: applying a colored cationic electrodeposition coating composition (A) to a metal substrate by electrodeposition; applying a clear coating composition (B) containing a bright pigment to the surface of the electrodeposition coating, which is either uncured or thermally cured; and curing the uncured electrodeposition coating and the clear coating, or the clear coating.

2. A method according to Item 1, wherein a base resin of the cationic electrodeposition coating composition (A) is at least one resin selected from the group consisting of cationic acrylic resins and cationic acrylic-modified epoxy resins.

3. A method according to Item 2, wherein the base resin of the cationic electrodeposition coating composition (A) is a cationic acrylic resin obtained by radical copolymerization of monomer components including a hydroxyl-containing acrylic monomer, an amino-containing acrylic monomer and an aromatic vinyl monomer.

4. A method according to Item 3, wherein the cationic acrylic resin has a hydroxyl value of about 10 to about 300 mg KOH/g, an amine value of about 10 to about 45 mg KOH/g, and a number average molecular weight of about 2,000 to about 100,000.

5. A method according to Item 2, wherein the base resin of the cationic electrodeposition coating composition (A) is a cationic acrylic-modified epoxy resin prepared by: reacting, with an epoxy resin, a carboxyl-containing acrylic resin obtained by radical copolymerization of monomer components including an α,β-ethylenically unsaturated carboxylic acid and a hydroxyl-containing acrylic monomer, followed by reaction with an amine compound.

6. A method according to Item 5, wherein the carboxyl-containing acrylic resin has a hydroxyl value of about 30 to about 200 mg KOH/g, an acid value of about 1 to about 50 mg KOH/g, and a number average molecular weight of about 2,000 to about 10,000.

7. A method according to Item 5, wherein the epoxy resin has a number average molecular weight of about 340 to about 3,000.

8. A method according to Item 5, wherein the proportions of the carboxyl-containing acrylic resin and the epoxy resin are about 90 to about 10% by weight of the former and about 10 to about 90% by weight of the latter, based on the total amount of the two resins.

9. A method according to Item 5, wherein the proportion of the amine compound is about 5 to about 35 parts by weight per 100 parts by weight of the total amount of the carboxyl-containing acrylic resin and the epoxy resin.

10. A method according to Item 1, wherein the bright pigment in the clear coating composition (B) is at least one member selected from the group consisting of metallic pigments and pearl pigments.

11. A method according to Item 1, wherein the clear coating composition (B) is applied to the surface of the uncured cationic electrodeposition coating, and then the uncured electrodeposition coating and the clear coating are thermally cured simultaneously.

12. A method according to Item 1, wherein the clear coating composition (B) is applied to the surface of the thermally cured electrodeposition coating, and then the uncured clear coating is cured thermally or by irradiation with active energy rays.

As used herein, “pearly luster” means a rainbow-like luster that exhibits various colors depending on the direction from which it is seen.

### DETAILED DESCRIPTION OF THE INVENTION

The method of the invention comprises: applying a colored cationic electrodeposition coating composition (A) to a metal substrate by electrodeposition; applying a clear coating composition (B) containing a bright pigment to the electrodeposition coating, which is either uncured or thermally cured; and curing the coating(s) to form a multilayer coating film comprising two layers (an electrodeposition coating layer and a clear coating layer).

Metal Substrate

The metal substrate may be made of a metal, such as iron-based, aluminium-based, zinc-based, copper-based, magnesium-based and like metals.

Specific examples of metal substrates include metal sheets and plates, formed or processed articles of metal sheets and plates, and the like.

The metal sheets and plates include, for example, sheets of carbon steel, stainless steel, high-strength steel, plated carbon steel and the like, aluminum sheets, aluminum alloy sheets, magnesium alloy sheets and the like. Among them, plated carbon steel sheets are preferable from the viewpoints of corrosion resistance and low cost. Examples of plated carbon steel sheets include hot-dip galvanized steel sheets, electrogalvanized steel sheets, electrogalvanized and iron-coated steel sheets, organic composite-plated steel sheets and the like.

Examples of pre-formed or processed articles of metal sheets or plates include automobile bodies, metal parts for two-wheeled vehicles, household electrical appliances, furniture and the like.

The above metal substrates may be cleaned by alkali degreasing or a similar process and then surface-treated by phosphate conversion, chromate conversion or like process.

Colored Cationic Electrodeposition Coating Composition (A)

The colored cationic electrodeposition coating composition (A) may be any known thermosetting coating composition comprising a base resin, a curing agent and a coloring pigment, and optionally containing an extender pigment, a surfactant or the like.

The coating composition (A) can be prepared by, for example, mixing an aqueous resin emulsion containing a base resin and a curing agent, with an aqueous pigment paste containing a coloring pigment, which have been selected according to the desired film performance.

The base resin for use in the coating composition (A) is preferably a cationic acrylic resin and/or a cationic acrylic-modified epoxy resin, from the viewpoint of weather resistance. A cationic epoxy resin, such as an amine-containing epoxy resin, can be additionally used as a base resin, in order to improve the corrosion resistance of the electrodeposition coating.

The cationic acrylic resin for use as a base resin of the coating composition (A) can usually be obtained by performing radical copolymerization of monomer components including a hydroxyl-containing acrylic monomer, an amino-containing acrylic monomer, an aromatic vinyl monomer and optionally other monomers, by a known process.

Examples of hydroxyl-containing acrylic monomers include 2-hydroxyethyl (meth)acrylate, 2-hydroxypropyl (meth)acrylate, 4-hydroxybutyl (meth)acrylate, adducts of 2-hydroxyethyl (meth)acrylate with caprolactone, and the like. Examples of such adducts include commercially available products, such as “Placcel FA-2” and “Placcel FM-3”. These monomers can be used either singly or in combination.

Examples of amino-containing acrylic monomers include N,N-dimethylaminoethyl (meth)acrylate, N,N-diethylaminoethyl (meth)acrylate, N,N-dimethylaminopropyl (meth)acrylate, N,N-di-t-butylaminoethyl (meth)acrylate, N,N-dimethylaminopropyl (meth)acrylamide and the like. These monomers can be used either singly or in combination.

Examples of aromatic vinyl monomers include styrene, vinyltoluene, α-methylstyrene and the like. These monomers can be used either singly or in combination.

Examples of other monomers include methyl (meth)acrylate, ethyl (meth)acrylate, n-propyl (meth)acrylate, isopropyl (meth)acrylate, n-butyl (meth)acrylate, isobutyl (meth)acrylate, t-butyl (meth)acrylate, cyclohexyl (meth)acrylate, 2-ethylhexyl (meth)acrylate and the like. These monomers can be used either singly or in combination.

To impart water solubility or water dispersibility, an adduct obtained by epoxy ring opening reaction of glycidyl (meth)acrylate and an active hydrogen-containing amine compound may be used in addition to the amino-containing acrylic monomer.

Examples of active hydrogen-containing amine compounds include primary mono- or polyamines, secondary mono- or polyamines, mixed primary/secondary polyamines, ketimine-blocked primary amino group-containing secondary mono- or polyamines, and ketimine-blocked primary amino group-containing hydroxyl compounds. Among these amine compounds, diethylamine, diethanolamine and the like are preferred. Ketimine-blocked forms of amine compounds (e.g., diethylenetriamine) are also preferred.

It is usually preferable that the cationic acrylic resin have a hydroxyl value of about 10 to about 300 mg KOH/g, in particular about 50 to about 200 mg KOH/g, an amine value of about 10 to about 45 mg KOH/g, in particular about 20 to about 40 mg KOH/g, and a number average molecular weight of about 2,000 to about 100,000, in particular about 3,000 to about 50,000.

A hydroxyl value less than 10 mg KOH/g results in a reduced crosslinking density in the electrodeposition coating layer, whereas a hydroxyl value exceeding 300 mg KOH/g results in lowered adhesion of the coating layer. Thus, hydroxyl values outside the specified range are undesirable. An amine value less than 10 mg KOH/g impairs the water dispersibility of the resin, whereas an amine value exceeding 45 mg KOH/g reduces the corrosion resistance of the coating layer. Thus, amine values outside the specified range are undesirable. A number average molecular weight less than 2,000 also reduces the corrosion resistance of the coating layer, whereas a number average molecular weight exceeding 100,000 deteriorates the finish properties of the coating layer. Therefore, number average molecular weights outside the specified range are undesirable.

The cationic acrylic-modified epoxy resin for use as a base resin of the cationic electrodeposition coating composition (A) can usually be obtained by Method I, which comprises: performing radical copolymerization of monomer components including α,β-ethylenically unsaturated carboxylic acid, a hydroxyl-containing acrylic monomer and optionally other monomers, to prepare a carboxyl-containing acrylic resin; reacting the acrylic resin with an epoxy resin to prepare an acrylic-modified epoxy resin; and reacting the acrylic-modified epoxy resin with an amine compound to render the resin cationic.

Examples of the α,β-ethylenically unsaturated carboxylic acid for use as a monomer component of the carboxyl-containing acrylic resin include acrylic acid, methacrylic acid, crotonic acid, itaconic acid, maleic acid, fumaric acid and the like. These monomers can be used either singly or in combination.

Examples of hydroxyl-containing acrylic monomers include those mentioned above.

Examples of the other monomers include methyl (meth)acrylate, ethyl (meth)acrylate, n-propyl (meth)acrylate, isopropyl (meth)acrylate, n-butyl (meth)acrylate, isobutyl (meth)acrylate, t-butyl (meth)acrylate, cyclohexyl (meth)acrylate, 2-ethylhexyl (meth)acrylate, styrene and the like. These monomers can be used either singly or in combination.

The carboxyl-containing acrylic resin preferably has a hydroxyl value of about 30 to about 200 mg KOH/g, in particular about 50 to about 150 mg KOH/g, an acid value of about 1 to about 50 mg KOH/g, in particular about 10 to about 30 mg KOH/g, and a number average molecular weight of about 2,000 to about 10,000, in particular about 5,000 to about 8,000.

A hydroxyl value exceeding 200 mg KOH/g reduces the water resistance and corrosion resistance of the coating layer, whereas a hydroxyl value less than 30 mg KOH/g lowers the crosslinking density in the coating layer, leading to reduced weather resistance and corrosion resistance. Thus, hydroxyl values outside the specified range are undesirable. An acid value less than 1 mg KOH/g reduces the reactivity with the epoxy resin, whereas an acid value exceeding 50 mg KOH/g is liable to cause gelation during the reaction with the epoxy resin. Therefore, acid values outside the specified range are undesirable. A number average molecular weight less than 2,000 results in insufficient acrylic modification of the epoxy resin, whereas a number average molecular weight over 10,000 is liable to cause gelation during the reaction with the epoxy resin. Therefore, number average molecular weights outside the specified range are undesirable.

A compound containing at least two epoxy groups per molecule and having a number average molecular weight of about 340 to about 3,000, preferably about 400 to about 3,000, more preferably about 800 to about 1,700 can be used as the epoxy resin to be reacted with the carboxyl-containing acrylic resin. When the epoxy resin has a number average molecular weight less than 340, the coating layer has insufficient corrosion resistance, whereas when the epoxy resin has a number average molecular weight exceeding 3,000, the surface smoothness of the coating layer is impaired. Thus, number average molecular weights outside the above range are undesirable.

Preferred as the epoxy resin is, for example, one obtained by the reaction of a polyphenol compound with epichlorohydrin. Examples of polyphenol compounds include bis(4-hydroxyphenyl)-2,2-propane, 4,4-dihydroxybenzophenone, bis(4-hydroxyphenyl)-1,1-isobutane, bis(4-hydroxy-t-butylphenyl)-2,2-propane, bis(2-hydroxynaphthyl)methane, tetra(4-hydroxyphenyl)-1,1,2,2-ethane, 4,4-dihydroxydiphenyl sulfone, phenol novolak, cresol novolac and the like.

The proportions of the carboxyl-containing acrylic resin and the epoxy resin used in the reaction to prepare an acrylic-modified epoxy resin are about 90 to about 10% by weight of the former and about 10 to about 90% by weight of the latter, based on the total amount of the two resins.

When the proportion of the acrylic resin is larger (i.e., when the proportion of the epoxy resin is smaller) than the above range, the electrodeposition coating layer has reduced corrosion resistance. On the other hand, when the proportion of the acrylic resin is smaller (i.e., when the proportion of the epoxy resin is larger) than the above range, the electrodeposition coating layer has reduced weather resistance. Thus, proportions outside the specified range are undesirable.

Next, an amine compound is reacted with the acrylic-modified epoxy resin to prepare a cationic acrylic-modified epoxy resin.

The proportion of the amine compound used in the reaction is about 5 to about 35 parts by weight, in particular about 10 to about 20 parts by weight, per 100 parts by weight of the total amount of the carboxyl-containing acrylic resin and epoxy resin that are the starting compounds for the acrylic-modified epoxy resin.

When the proportion of the amine compound is less than 5 parts by weight, the resin has decreased water dispersibility, whereas when the proportion exceeds 35 parts by weight, the electrodeposition coating layer is insufficient in corrosion resistance and weather resistance.

Examples of amine compounds include diethylamine, dibutylamine, methylbutylamine, diethanolamine and the like. Also usable are ketimine-blocked forms of amine compounds such as diethylenetriamine. They may be used either singly or in combination.

The cationic acrylic-modified epoxy resin for use as a base resin of the cationic electrodeposition coating composition (A) can be obtained also by Method II, which comprises: performing radical copolymerization of a mixture containing an α,β-ethylenically unsaturated group-containing epoxy resin obtained by reacting an epoxy resin with an α,β-ethylenically unsaturated carboxylic acid, a hydroxyl-containing acrylic monomer, and optionally other monomers; and reacting the resulting copolymer with an amine compound to render the resin cationic.

Method II is the same as Method I except that α,β-ethylenically unsaturated carboxylic acid is reacted with the epoxy resin in advance, instead of reacting the carboxyl-containing acrylic resin with the epoxy resin. Therefore, the epoxy resin, α,β-ethylenically unsaturated carboxylic acid, hydroxyl-containing acrylic monomer, other monomers and amine compound for use as starting materials in Method II may be the same as those for use in Method I.

In order to improve the corrosion resistance of the electrodeposition coating layer, a cationic epoxy resin, such as an amine-containing epoxy resin, can be additionally used as a base resin.

The amine-containing epoxy resin may be one conventionally used in electrodeposition coating compositions, such as (i) an adduct of a polyepoxide compound with a primary mono- or polyamine, a secondary mono- or polyamine, or a mixed primary/secondary polyamine, (ii) an adduct of a polyepoxide compound with a ketimine-blocked primary amino group-containing secondary mono- or polyamine, (iii) a reaction product obtained by the etherification of a polyepoxide compound with a ketimine-blocked primary amino group-containing hydroxy compound.

The adduct (i) is described in, for example, U.S. Pat. No. 3,984,299. The adduct (ii) is described in, for example, U.S. Pat. No. 4,017,438. The reaction product (iii) is described in, for example, Japanese Unexamined patent Publication No. 1984-43013.

Usually, a blocked polyisocyanate compound is suitable as a curing agent for the cationic electrodeposition coating composition (A). Especially preferred as the blocked polyisocyanate compound is a compound obtained by blocking, with a blocking agent, at least one member selected from alicyclic polyisocyanate compounds and aliphatic polyisocyanate compounds, from the viewpoints of weather resistance and yellowing resistance.

Examples of the polyisocyanate compounds include alicyclic diisocyanate compounds, such as isophorone diisocyanate, dicyclohexylmethane-4,4′-diisocyanate and cyclohexylene diisocyanate; aliphatic diisocyanate compounds, such as hexamethylene diisocyanate, tetramethylene diisocyanate and methylene diisocyanate; dimers and trimers of these diisocyanate compounds; isocyanate-terminated compounds obtained by reacting a low molecular weight active hydrogen-containing compound with an excessive amount of any of these diisocyanate compounds.

Examples of low molecular weight active hydrogen-containing compounds include ethylene glycol, propylene glycol, trimethylolpropane, hexanetriol, castor oil and the like.

The blocking agent undergoes addition to isocyanate groups of the polyisocyanate compound and blocks the isocyanate groups. Preferably, the blocked polyisocyanate compound produced by addition is stable at room temperature, and capable of recovering the isocyanate groups by dissociation of the blocking agent when heated at about 100 to about 200° C.

Blocking agents satisfying the above conditions include, for example, ε-caprolactam, γ-caprolactam and other lactam compounds; methyl ethyl ketoxime, cyclohexanone oxime and other oxime compounds; phenylcarbinol, methylphenylcarbinol and other aromatic alkyl alcohols; ethylene glycol monobutyl ether and other ether alcohol compounds; and the like.

The aqueous resin emulsion containing a base resin and a curing agent can be usually obtained by adding, as required, additives, such as a surfactant, a surface modifier and a curing catalyst, to a mixture of the base resin and curing agent, and then adding a neutralizing agent, such as an aliphatic carboxylic acid, to neutralize and disperse the base resin. Examples of aliphatic carboxylic acids include acetic acid, formic acid, lactic acid and other water soluble organic acids.

The aqueous pigment paste containing a coloring pigment can be obtained usually by mixing a coloring pigment, an extender pigment, a rust preventive pigment, a curing catalyst or the like with a dispersing resin, its neutralizer and deionized water, followed by stirring and dispersion using a ball mill, a sand mill or the like.

Examples of coloring pigments include, but are not limited to, the following organic or inorganic coloring pigments.

White pigments: titanium white, zinc white, lithopone, zinc sulfide, antimony white, etc.

Black pigments: carbon black, acetylene black, lampblack, graphite, iron black, aniline black, etc.

Yellow pigments: ocher, yellow iron oxide, naphthol yellow S, Hansa yellow 10G, Hansa yellow 5G, Hansa yellow 3G, Hansa yellow G, Hansa yellow GR, Hansa yellow A, Hansa yellow RN, Hansa yellow R, pigment yellow L, benzidine yellow, benzidine yellow G, benzidine yellow GR, permanent yellow NCG, vulcan fast yellow 5G, vulcan fast yellow R, tartrazine lake, quinoline yellow lake, anthragen yellow 6GL, etc.

Orange pigments: chrome orange, chrome vermilion, Sudan I, permanent orange, lithol fast orange 3GL, permanent orange GTR, Hansa yellow 3R, vulcan fast orange GG, benzidine orange G, Persian orange, indathrene brilliant orange GK, indathrene brilliant orange RK, etc.

Brown pigments: iron oxide, umber, etc.

Red pigments: red iron oxide, permanent red 4R, permanent red F5R, para red, fire red, parachloro orthonitro aniline red, lithol fast scarlet G, brilliant fast scarlet, brilliant carmine BS, brilliant carmine 6B, permanent red F2R, permanent red F4R, permanent red FRL, permanent red FRLL, permanent red F4RH, fast scarlet VD, vulcan fast rubine B, vulcan fast pink G, light fast red toner B, light fast red toner R, permanent carmine FB, lake red, anthosine B, brilliant scarlet G, lithol rubine GK, pigment scarlet 3B, Bordeaux 5B, toluidine maroon, permanent Bordeaux F2R, Helio Bordeaux BL, Bordeaux 10B, bon maroon light, bon maroon medium, eosin lake, rhodamine lake B, rhodamine lake Y, alizarin lake, thioindigo red B, thioindigo maroon, quinacridone red pigment, etc.

Purple pigments: cobalt purple, manganese purple, fast violet B, methyl violet lake, etc.

Blue pigments: ultramarine blue, Prussian blue, cobalt blue, cerulean blue, nonmetallic phthalocyanine blue, phthalocyanine blue, fast sky blue, indathrene blue RS, indathrene blue BC, indigo, etc.

Green pigments: chrome green, pigment green B, naphthol green B, green gold, phthalocyanine green, etc.

Examples of extender pigments include kaolin, baryta powder, precipitated barium sulfate, barium carbonate, calcium carbonate, gypsum, clay, silica, white carbon, diatomaceous earth, talc, magnesium carbonate, alumina white, gloss white, mica powder and the like.

Examples of rust preventive pigments include aluminum tripolyphosphate, zinc tripolyphosphate, zinc white, inorganic bismuth, organic acid bismuth and the like.

Examples of curing catalysts include dibutyltin oxide (DBTO), dioctyltin oxide (DOTO), dibutyltin dibenzoate and other tin catalysts.

Examples of dispersing resins include tertiary amine epoxy resins, quaternary ammonium salt epoxy resins, tertiary amine acrylic resins and the like.

Examples of neutralizers include acetic acid, formic acid, lactic acid and the like.

The colored cationic electrodeposition coating composition (A) can be obtained by adding deionized water to the mixture of the aqueous resin emulsion and the aqueous pigment paste, and if required, further adding additives, such as a surfactant, a surface modifier and a pH adjustor, followed by stirring and mixing. It is suitable that the electrodeposition coating composition have a solid content of about 5 to about 25% by weight, and a pH of about 5 to about 8. The composition can be used as an electrodeposition bath for electrodeposition coating.

A suitable mixing ratio of the aqueous resin emulsion and the aqueous pigment paste is usually such that the amount of the pigments including a coloring pigment is about 0.1 to about 70 parts by weight, preferably about 5 to about 40 parts by weight, per 100 parts by weight of the resin solids.

Clear Coating Composition (B) Containing Bright Pigment

Various coating compositions, such as organic solvent-based, aqueous or powdery coating compositions, are usable as the clear coating composition (B) without limitation, as long as they are bright pigment-containing clear coating compositions with good weather resistance. Various resins, such as acrylic resins, polyester resins, alkyd resins, silicone resins and fluororesins, are usable as a base resin of the clear coating composition. The base resin may be a thermosetting resin to be used in combination with a curing agent, or a resin curable by active energy rays, such as ultraviolet rays or an electron beam.

Preferred examples of the clear coating composition (B) include a thermosetting coating composition comprising: a base resin (e.g., an acrylic resin, a polyester resin or an alkyd resin) having hydroxyl groups or like crosslinkable functional groups; a curing agent reactive with the crosslinkable functional groups, such as a melamine resin, a urea resin, a polyisocyanate compound or a blocked polyisocyanate; and a bright pigment.

Other preferred examples of the clear coating composition (B) include a thermosetting coating composition comprising an epoxy- and hydroxyl-containing acrylic copolymer as a base resin, a carboxyl-containing compound as a curing agent, and a bright pigment. The copolymer can be obtained, for example, by radical polymerization of an epoxy-containing acrylic monomer (e.g., glycidyl (meth)acrylate), a hydroxyl-containing radically polymerizable monomer, and optionally other monomers.

The bright pigment is at least one member selected from the group consisting of metallic pigments and pearl pigments. Examples of metallic pigments include aluminium flakes, deposited aluminium thin films, aluminium oxide flakes and the like. Examples of pearl pigments include mica flakes, titanium oxide-coated mica flakes, iron oxide-coated mica flakes and the like.

The bright pigment is added preferably in such an amount that the underlying colored cationic electrodeposition coating layer can be seen through the clear coating layer. A suitable amount of the bright pigment to be added is about 0.001 to about 5 parts by weight, preferably about 0.01 to about 2 parts by weight, more preferably about 0.02 to about 1 parts by weight, per 100 parts by weight of the resin solids in the clear coating composition (B).

The clear coating composition (B) can be prepared by mixing a base resin, a bright pigment, and optionally additives such as a curing agent, an ultraviolet absorber and a surface modifier, in water and/or an organic solvent. It is desirable that the clear coating composition have, at the time of application, a resin solid content of 20 to 60% by weight, preferably 25 to 50% by weight, and a viscosity of about 10 to about 30 seconds/Ford cup #4/20° C.

Steps for Forming Multilayer Coating Film

In the method of the invention, the colored cationic electrodeposition coating composition (A) is applied to a metal substrate by electrodeposition and optionally cured by heating, and the clear coating composition (B) is applied, and then the uncured electrodeposition coating and the clear coating film are cured or the clear coating alone is cured, to form a multilayer coating film.

Therefore, the method of the invention may be: a 2-coat 1-bake method comprising applying the cationic electrodeposition coating composition (A) by electrodeposition, applying the clear coating composition (B) to the uncured electrodeposition coating, and then thermally curing the uncured electrodeposition coating and the clear coating simultaneously; or a 2-coat 2-bake method comprising applying the cationic electrodeposition coating composition (A) by electrodeposition, thermally curing the electrodeposition coating, applying the clear coating composition (B) to the thermally cured electrodeposition coating, and then curing the uncured clear coating thermally or by irradiation with active energy rays.

When the clear coating composition (B) is curable by activity energy rays, the multilayer coating film is formed by the 2-coat 2-bake method.

Known electrodeposition coating processes and equipment can be employed to apply the coating composition (A) to a metal substrate by cationic electrodeposition.

The conditions for electrodeposition are not limited, but it is generally preferable to conduct electrodeposition with stirring under the following conditions: a bath temperature of 15 to 35° C., preferably 20 to 30° C., a voltage of 100 to 400 V, preferably 200 to 300 V, an energization time of 30 seconds to 10 minutes, an anode/cathode area ratio of 8/1 to 1/8, and an anode-cathode distance of 10 to 200 cm.

The thickness of the cationic electrodeposition coating layer is suitably selected according to the desired performance, but it is usually suitable that the coating layer be about 5 to about 60 μm thick, preferably about 10 to about 40 μm thick, when cured.

After the electrodeposition, extra cationic electrodeposition coating composition is removed by thorough washing with ultrafiltrate, industrial water, pure water or the like, so that no extra cationic electrodeposition coating composition remains on the coated substrate.

To form the coating film by the 2-coat 1-bake method, the electrodeposition coating is set at room temperature or preheated at about 40 to about 110° C. for about 10 to about 180 minutes, before applying the clear coating composition (B).

To form the coating film by the 2-coat 2-bake method, the electrodeposition coating is cured by heating the surface of the coated substrate to about 110 to about 200° C., preferably 140 to 180° C., for about 10 to about 180 minutes, preferably 20 to 50 minutes, using an electric hot air dryer, a gas hot air dryer or the like, before applying the clear coating composition (B).

The clear coating composition (B) is applied to the cured or uncured surface of the colored cationic electrodeposition coating by air spray, airless spray, electrostatic coating or like coating process, so that the clear coating layer is at least 15 μm thick, preferably 20 to 70 μm thick, when cured.

Subsequently, the uncured electrodeposition coating and clear coating are cured simultaneously, or the uncured clear coating alone is cured, to form a multilayer coating film. When the clear coating composition (B) is thermosetting, curing of both the electrodeposition coating and the clear coating or the clear coating alone is carried out by heating at about 80 to about 170° C. for about 10 to about 40 minutes, thereby forming a multilayer coating film. When the clear coating composition (B) is curable by active energy rays, the clear coating is cured by irradiation with about 100 to about 2,000 mJ/cm², preferably about 500 to about 1,500 mJ/cm², of active energy rays, such as ultraviolet rays or an electron beam, so that a multilayer coating film is formed.

In this manner, a multilayer coating film comprising a colored cationic electrodeposition coating layer and a bright pigment-containing clear coating layer on the electrodeposition coating layer is formed on a metal substrate. The multilayer coating film offers new three-dimensional design with attractive visual effects, such as three-dimensional brightness, glitter and/or pearly luster, and is excellent in weather resistance, corrosion resistance and finish properties.

### EXAMPLES

The following Production Examples, Examples and Comparative Examples are provided to illustrate the present invention in further detail. In these examples, parts and percentages are all by weight.

### Production Example 1

Production of Aqueous Pigment Paste A

An aqueous dispersion resin with a solid content of 85% (5.88 parts (5 parts as solids)) obtained by acid neutralization of a tertiary amine epoxy resin, and 10% acetic acid (2.7 parts) were mixed together. Then, 25.8 parts of deionized water was further added, followed by mixing and stirring. Titanium white (20 parts), deionized water (16.1 parts), bismuth oxide (2 parts) and dibutyltin oxide (1 part) were added, and the resulting mixture was dispersed for 20 hours using a ball mill, giving 50.9 parts of aqueous pigment paste A with a solid content of 55%.

### Production Examples 2 to 4

Production of Aqueous Pigment Pastes B to D

The production procedure for aqueous pigment paste A was repeated using the components shown in Table 1, to obtain aqueous pigment pastes B to D. The amounts of the components in Table 1 are indicated by part(s). The numerical values in the parentheses show the solid contents.

TABLE 1

| 55% aqueous pigment paste | | A | B | C | D |
|---|---|---|---|---|---|
| Color | | White | Blue | Red | Gray |
| 85% aqueous dispersion resin | | 5.88 | 5.88 | 5.88 | 5.88 |
| | | (5) | (5) | (5) | (5) |
| 10% acetic acid | | 2.7 | 2.7 | 2.7 | 2.7 |
| Deionized water | | 19.32 | 19.32 | 19.32 | 19.32 |
| Coloring | Titanium white | 20 | | | 14 |
| pigment | Carbon black | | 0.1 | 0.1 | 0.3 |
| | Copper | | 10 | | |
| | phthalocyanine blue | | | | |
| | Quinacridone Red | | | 10 | |

| | | | | |
|---|---|---|---|---|
| Purified clay | | 9.9 | 9.9 | 5.7 |
| Bismuth oxide | 2 | 2 | 2 | 2 |
| Dibutyltin oxide | 1 | 1 | 1 | 1 |
| Total amount | 50.9 | 50.9 | 50.9 | 50.9 |
| | (28.0) | (28.0) | (28.0) | (28.0) |

### Production Example 5

Production of Cationic Acrylic Resin

Propylene glycol monomethyl ether (246 parts) was placed in a 2-L four-necked flask, and after purging with nitrogen, maintained at 110° C. Into the flask, a mixture of styrene (25 parts), methyl methacrylate (18 parts), n-butyl acrylate (6 parts), 2-hydroxyethyl methacrylate (12 parts), “Placcel FM-3” (a tradename of Daicel Chemical Industries, Ltd.) (24 parts), dimethylaminoethyl methacrylate (15 parts) and azobisisobutyronitrile (3 parts) was added dropwise over 3 hours.

One hour after completion of the addition, a solution of 8 parts of 2,2′-azobis(2-methylbutyronitrile) in 56 parts of propylene glycol monomethyl ether was added dropwise over 1 hour. After completion of the addition, the resulting mixture was maintained at 110° C. for a further 1 hour, and then methyl isobutyl ketone was added, giving a cationic acrylic resin with a solid content of 75%. The cationic acrylic resin had a hydroxyl value of 130 mg KOH/g, an amine value of 26 mg KOH/g and a number average molecular weight of 8,000.

### Production Example 6

Production of Curing Agent

Isophorone diisocyanate (IPDI) (50 parts) was added dropwise at 40 to 60° C. to methyl ethyl ketoxime (40 parts) in a 4-L flask. The mixture in the flask was heated at 80° C. for 1 hour to add methyl ethyl ketoxime to the isocyanate groups of IPDI, giving a curing agent for cationic electrodeposition coating compositions. The curing agent had a solid content of 90%.

### Production Example 7

Production of Aqueous Resin Emulsion

The cationic acrylic resin with a solid content of 75% obtained by Production Example 5 (93.3 parts (70 parts as solids)), the curing agent obtained in Production Example 6 (33.3 parts (30 parts as solids)), a 40% butyl cellosolve solution of dibutyltin dibenzoate (2.5 parts), and 10% formic acid (8.2 parts) were mixed and stirred uniformly. Thereafter, 178.3 parts of deionized water was added dropwise over about 15 minutes with strong agitation, giving an aqueous resin emulsion with a solid content of 32.0%.

### Production Example 8

Production of Cationic Electrodeposition Coating Composition

The 32% aqueous resin emulsion obtained in Production Example 7 (315.6 parts (101 parts as solids)), 55% aqueous pigment paste A obtained in Production Example 1 (70 parts (38.5 parts as solids)), and pure water (311.9 parts) were mixed together, giving cationic electrodeposition coating composition No. 1 with a solid content of 20%.

### Production Examples 9 to 11

Production of Cationic Electrodeposition Coating Composition

Using the components shown in Table 2, cationic electrodeposition coating compositions No.2 to No.4 were produced. The amounts of the components in Table 1 are indicated by parts. The numerical values in the parentheses show the solid contents.

TABLE 2

| 20% cationic | | | | | |
|---|---|---|---|---|---|
| electrodeposition | | | | | |
| coating composition | | No. 1 | No. 2 | No. 3 | No. 4 |
| Color | | White | Blue | Red | Gray |
| 32% aqueous resin | | 315.6 | 315.6 | 315.6 | 315.6 |
| emulsion | | (101) | (101) | (101) | (101) |
| 55% | Type | A | B | C | D |
| aqueous | Amount | 70 | 70 | 70 | 70 |
| pigment | | (38.5) | (38.5) | (38.5) | (38.5) |
| paste | | | | | |

| | | | | |
|---|---|---|---|---|
| Deionized water | 311.9 | 311.9 | 311.9 | 311.9 |
| Total amount | 697.5 | 697.5 | 697.5 | 697.5 |
| | (139.5) | (139.5) | (139.5) | (139.5) |

### Production Example 12

Production of Aqueous Acrylic Resin Dispersion

n-Butyl acrylate (16.7 parts), methyl methacrylate (15 parts), styrene (30 parts), 2-ethylhexyl acrylate (20 parts), hydroxyethyl methacrylate (12 parts), acrylic acid (6.3 parts) and azobisisobutyronitrile (1 part) were added to butyl cellosolve maintained at 115° C. in a 4-L flask. Then, polymerization reaction was conducted under ordinary conditions to synthesize a carboxyl- and hydroxyl-containing acrylic resin.

The obtained acrylic resin had an acid value of 50 mg KOH/g, a hydroxyl value of 50 mg KOH/g and a number average molecular weight of 45,000. The carboxyl groups of the acrylic resin were neutralized with an equivalent of dimethylaminoethanol, giving an aqueous acrylic resin dispersion with a solid content of 55%.

### Production Example 13

Production of Aqueous Melamine Resin Dispersion

Melamine (126 parts), 80% paraformaldehyde (manufactured by Mitsui Chemicals, Inc.) (225 parts) and n-butanol (592 parts) were placed in a 2-L four-necked flask equipped with a thermometer, a stirrer and a reflux condenser. The mixture in the flask was adjusted to pH 9.5 to 10.0 with a 10% aqueous sodium hydroxide solution, and reacted at 80° C. for 1 hour. Thereafter, 888 parts of n-butanol was added, and the resulting mixture was adjusted to pH 5.5 to 6.0 with a 5% sulfuric acid solution and reacted at 80° C. for 3 hours. After completion of the reaction, the reaction mixture was neutralized to pH 7.0 to 7.5 with a 20% aqueous sodium hydroxide solution. N-butanol was distilled off under reduced pressure at 60 to 70° C., and the residue was filtered to obtain an aqueous dispersion of a butyl-etherified melamine resin with a solid content of 27%. The melamine resin was hydrophobic, and had a dilution ratio of 3.6% with a water/methanol mixed solvent (35/65 in a weight ratio), and a weight average molecular weight of 800.

Twenty-five parts (as solids) of the melamine resin was weighed out into a stirring vessel. Into the vessel, 20 parts of a 50% aqueous acrylic resin solution was placed, and then 80 parts of deionized water was gradually added while stirring with a disper mixer at 1,000 to 1,500 rpm. Stirring was continued for a further 30 minutes, giving an aqueous melamine resin dispersion with a solid content of 27%.

The 50% aqueous acrylic resin solution was an aqueous solution of an acrylic resin with a hydroxyl value of 14.5 mg KOH/g and a number average molecular weight of 8,000, obtained by copolymerizing, in a routine manner, a monomer mixture consisting of styrene (30 parts), methyl methacrylate (20 parts), 2-ethylhexyl methacrylate (18 parts), “Placcel FM-3” (a tradename of Daicel Chemical Industries, Ltd., an adduct of 2-hydroxyethyl methacrylate and caprolactone) (10 parts) and acrylic acid (4 parts).

### Production Example 14

Production of Clear Coating Composition

A mixture of the aqueous acrylic resin dispersion with a solid content of 55% obtained in Production Example 12 (163 parts), the aqueous melamine resin dispersion with a solid content of 27% obtained in Production Example 13 (131 parts), a thickener (Note 1) (3 parts), an ultraviolet absorber (Note 2) (1 part), dimethylaminoethanol (0.3 parts), deionized water (171 parts) and a metallic pigment (Note 3) (0.5 parts) was adjusted to a viscosity of 30 seconds (Ford cup #4/20° C.) with deionized water, giving metallic pigment-containing aqueous clear coating composition No. 1 with a solid content of 40%.

Notes 1 to 3 are as follows.

Note 1: “Primal ASE-60” (a tradename of Rohm-and-Haas, a thickener)

Note 2: “Tinuvin 900” (a tradename of Ciba-Geigy, an ultraviolet absorber)

Note 3: “Aluminium Paste 891K” (a tradename of Toyo Aluminium K.K., an aluminium flake pigment paste with an aluminum content of 72%)

### Production Example 15

Production of Pearl Pigment Paste

A pearl pigment (Note 4) (0.5 parts), a phosphoric acid group-containing acrylic resin (Note 5) (1.6 parts) and butyl cellosolve (16 parts) were mixed together, with a disper mixer. Then, 6 parts of the 55% aqueous acrylic resin dispersion obtained in Production Example 12 was added, followed by further stirring with the disper mixer to thereby obtain a pearl pigment paste with a solid content of 55%.

Notes 4 and 5 are as follows.

Note 4: “Pearl Mica White” (a tradename of Marc, iron oxide-coated mica with a thickness of 0.2 to 0.5 μm)

Note 5: A phosphoric acid group-containing acrylic resin with a hydroxyl value of 29.2 mg KOH/g and a number average molecular weight of 10,000, obtained by copolymerizing, in a routine manner, a monomer mixture consisting of styrene (25 parts), 2-ethylhexyl methacrylate (27.5 parts), 2-hydroxyethyl methacrylate (20 parts), 4-hydroxybutyl acrylate (7.5 parts), a 50% phosphoric acid group-containing polymerizable monomer (15 parts) and 2-methacryloyloxyethyl acid phosphate (12.5 parts). The 50% phosphoric acid group-containing polymerizable monomer is a monomer obtained by reacting and aging, with stirring, monobutylphosphoric acid (57.5 parts), isobutanol (41.1 parts), glycidyl methacrylate (42.5 parts) and isopropanol (58.9 parts).

### Production Example 16

Production of Clear Coating Composition

A mixture of the pearl pigment paste obtained in Production Example 15 (24.1 parts), the aqueous acrylic resin dispersion with a solid content of 55% obtained in Production Example 12 (162 parts), the aqueous melamine resin dispersion with a solid content of 27% obtained in Production Example 13 (131 parts), a thickener (Note 1) (3 parts), an ultraviolet absorber (Note 2) (1 part), dimethylaminoethanol (0.3 parts) and deionized water (147 parts) was adjusted to a viscosity of 30 seconds (Ford cup #4/20° C.) with deionized water, to thereby obtain a pearl pigment-containing aqueous clear coating composition No. 2 with a solid content of 40%.

Notes 1 to 3 are the same as above.

### Example 1

Cationic electrodeposition coating composition No.1 obtained in Production Example 8 was applied by electrodeposition to a cold-rolled steel sheet which had been subjected to zinc phophate treatment using “Palbond #3020” (a tradename of Nihon Parkerizing Co., Ltd., a zinc phosphate treating agent), to a thickness of 20 μm (when cured), and heated at 150° C. for 20 minutes for curing.

Aqueous clear coating composition No.1 obtained in Production Example 14 was applied to the cured electrodeposition coating by spray coating to a thickness of 40 μm (when cured), and heated at 140° C. for 20 minutes for curing, to thereby form a multilayer coating film of Example 1.

### Examples 2 to 7 and Comparative Examples 1 to 5

Multilayer coating films of Examples 2 to 7 and Comparative Examples 1 to 5 were formed by following the procedure of Example 1, using the combinations of electrodeposition coating compositions and clear coating compositions shown in Table 3.

TABLE 3

| | Ex.1 | Ex.2 | Ex.3 | Ex.4 | Ex.5 | Ex.6 |
|---|---|---|---|---|---|---|
| Electrode- | No.1 | No.2 | No.3 | No.1 | No.2 | No.3 |
| position | | | | | | |
| coating | | | | | | |
| composition | | | | | | |
| Color | White | Blue | Red | White | Blue | Red |
| Curing | 150° C. | 150° C. | 150° C. | 150° C. | 150° C. | 150° C. |
| conditions | 20 min | 20 min | 20 min | 20 min | 20 min | 20 min |
| Clear coating | No.1 | No.1 | No.1 | No.2 | No.2 | No.2 |
| composition | | | | | | |
| Color | Metalic | Metalic | Metalic | Pearl | Pearl | Pearl |
| Curing | 140° C. | 140° C. | 140° C. | 140° C. | 140° C. | 140° C. |
| conditions | 20 min | 20 min | 20 min | 20 min | 20 min | 20 min |
| | Comp. | Comp. | Comp. | Comp. | Comp. | |
| | Ex.7 | Ex.1 | Ex.2 | Ex.3 | Ex.4 | Ex.5 |
| Electrode- | No.1 | No.1 | No.2 | No.3 | No.4 | No.4 |
| position | | | | | | |
| coating | | | | | | |
| composition | | | | | | |
| Color | White | White | Blue | Red | Gray | Gray |
| Curing | 110° C. | 110° C. | 110° C. | 110° C. | 110° C. | 110° C. |
| conditions | 10 min | 10 min | 10 min | 10 min | 10 min | 10 min |
| Clear coating | No.1 | Note 6 | Note 6 | Note 6 | Note 6 | Note 6 |
| composition | | | | | | |
| Color | Metalic | Clear | Clear | Clear | Clear | Clear |
| Curing | 140° C. | 140° C. | 140° C. | 140° C. | 140° C. | 140° C. |
| conditions | 20 min | 20 min | 20 min | 20 min | 20 min | 20 min |

Note 6 in Table 3 is as follows.

Note 6: “Lugabake Clear” (a tradename of Kansai Paint Co., Ltd., an organic solvent-based acrylic resin/amino resin clear coating composition)

Film Performance Test

Performance tests were conducted by the following methods to evaluate the weather resistance, brightness and gloss of the multilayer films obtained in Examples 1 to 7 and Comparative Examples 1 to 5.

Weather resistance: The accelerated weathering test using a sunshine carbon arc lamp, defined in JIS K-5400 9.8.1, was conducted for an irradiation time of 1,000 hours. Thereafter, crosscuts were formed on the film surface of each test plate using a cutter, and an adhesive tape was attached to the crosscut portion and rapidly peeled off. The resulting coating films were checked for peeling. The evaluation criteria are as follows.

A: No peeling of the coating film, indicating good weather resistance;

B: Partial peeling of the coating film, indicating slightly poor weather resistance;

C: Peeling of the whole coating film, indicating poor weather resistance.

Brightness: The appearances of the multilayer coating films were visually evaluated. The evaluation criteria are as follows.

A: Remarkable metallic or pearly depth and three-dimensional appearance on the film surface.

B: Satisfactory metallic or pearly depth and three-dimensional appearance on the film surface.

C: Little metallic or pearly depth or three-dimensional appearance on the film surface.

D: No metallic or pearly depth or three-dimensional appearance on the film surface.

Gloss: The gloss was evaluated by the 60° specular reflectivity (%).

Table 4 presents the results of the film performance tests.

TABLE 4

| | Examples | | | | | | | Comp. Examples | | | | |
|---|---|---|---|---|---|---|---|---|---|---|---|---|
| | 1 | 2 | 3 | 4 | 5 | 6 | 7 | 1 | 2 | 3 | 4 | 5 |
| Weather | A | A | A | A | A | A | A | A | A | A | A | A |
| resistance | | | | | | | | | | | | |
| Brightness | A | A | A | A | A | A | B | C | C | C | C | D |
| Gloss | 96 | 96 | 96 | 96 | 96 | 96 | 94 | 93 | 93 | 93 | 93 | 91 |

The method for forming a multilayer coating film according to the invention has the following remarkable advantages.

(1) By the method of the invention, a multilayer coating film is formed on a metal substrate, the coating film comprising a colored cationic electrodeposition coating layer and a bright pigment-containing clear coating layer formed on the electrodeposition coating layer. The multilayer coating film has new three-dimensional design with attractive visual effects, such as three-dimensional brightness, glitter and/or pearly luster, and is excellent in weather resistance, corrosion resistance and finish properties.

Since the bright pigment-containing clear coating layer is formed on the colored electrodeposition coating layer, the reflected light from the colored coating layer is combined with the brightness, glitter, pearly luster, etc. from the bright pigment to produce the three-dimensional design with attractive visual effects.

(2) Accordingly, when a multilayer coating film is formed on a metal substrate by the method of the invention, the attractive visual effects are exhibited variously depending on the angle, intensity and other aspects of the light hit on, for example, horizontal, vertical or curved parts, specifically fenders, doors or hoods of automobile bodies.

1. A method for forming a multilayer coating film, comprising the steps of: applying a colored cationic electrodeposition coating composition (A) to a metal substrate by electrodeposition; applying a clear coating composition (B) containing a bright pigment to the surface of the electrodeposition coating, which is either uncured or thermally cured; and curing the uncured electrodeposition coating and the clear coating, or the clear coating.

2. A method according to claim 1, wherein a base resin of the cationic electrodeposition coating composition (A) is at least one resin selected from the group consisting of cationic acrylic resins and cationic acrylic-modified epoxy resins.

3. A method according to claim 2, wherein the base resin of the cationic electrodeposition coating composition (A) is a cationic acrylic resin obtained by radical copolymerization of monomer components including a hydroxyl-containing acrylic monomer, an amino-containing acrylic monomer and an aromatic vinyl monomer.

4. A method according to claim 3, wherein the cationic acrylic resin has a hydroxyl value of about 10 to about 300 mg KOH/g, an amine value of about 10 to about 45 mg KOH/g, and a number average molecular weight of about 2,000 to about 100,000.

5. A method according to claim 2, wherein the base resin of the cationic electrodeposition coating composition (A) is a cationic acrylic-modified epoxy resin prepared by: reacting, with an epoxy resin, a carboxyl-containing acrylic resin obtained by radical copolymerization of monomer components including an α,β-ethylenically unsaturated carboxylic acid and a hydroxyl-containing acrylic monomer, followed by reaction with an amine compound.

6. A method according to claim 5, wherein the carboxyl-containing acrylic resin has a hydroxyl value of about 30 to about 200 mg KOH/g, an acid value of about 1 to about 50 mg KOH/g, and a number average molecular weight of about 2,000 to about 10,000.

7. A method according to claim 5, wherein the epoxy resin has a number average molecular weight of about 340 to about 3,000.

8. A method according to claim 5, wherein the proportions of the carboxyl-containing acrylic resin and the epoxy resin are about 90 to about 10% by weight of the former and about 10 to about 90% by weight of the latter, based on the total amount of the two resins.

9. A method according to claim 5, wherein the proportion of the amine compound is about 5 to about 35 parts by weight per 100 parts by weight of the total amount of the carboxyl-containing acrylic resin and the epoxy resin.

10. A method according to claim 1, wherein the bright pigment in the clear coating composition (B) is at least one member selected from the group consisting of metallic pigments and pearl pigments.

11. A method according to claim 1, wherein the clear coating composition (B) is applied to the surface of the uncured cationic electrodeposition coating, and then the uncured electrodeposition coating and the clear coating are thermally cured simultaneously.

12. A method according to claim 1, wherein the clear coating composition (B) is applied to the surface of the thermally cured electrodeposition coating, and then the uncured clear coating is cured thermally or by irradiation with active energy rays.
